(19) United States
(12) Patent Application Publication
(10) Pub. No.: US 2013/0282054 A1
(43) Pub. Date: Oct. 24, 2013

(54) OCCLUSION DEVICE FOR UNDESIRED OPENINGS IN THE HEART

(71) Applicant: Peter Osypka, Rheinfelden (DE)

(72) Inventor: Peter Osypka, Rheinfelden (DE)

(21) Appl. No.: 13/978,907

(22) PCT Filed: Jan. 12, 2012
(86) PCT No.: PCT/EP2012/000120

§ 371 Date: Jul. 10, 2013

(30) Foreign Application Priority Data
Jan. 12, 2011 (DE) 20 2011 001 366.9

(51) Int. Cl. A61B 17/00

(52) U.S. Cl. CPC A61B 17/0057
USPC 606/213

(57) ABSTRACT

An occlusion device (100) for undesired openings in the heart (ASD, PFO, VSD) has two flexible occlusion disks (3), which are arranged at a distance from each other and which are made of a meshwork of metal wires. The occlusion device can be used to occlude, for example, openings between two atriums, especially in the human heart, wherein the occlusion device (100) can also be recovered and re-positioned if necessary or if the occlusion device is positioned incorrectly. The occlusion disks are connected to each other by welding at the points facing each other in the region of the smallest radial extension of the occlusion device (100), so that clamps, clips, and/or threads can be avoided.

### BACKGROUND

The invention relates to an occlusion device for undesired openings in the heart (ASD, PFO, VSD), with two flexible and welded umbrellas which are arranged at a distance from each other, serve as occlusion disks and are made from a braid of metal wires, whereby openings in the human heart can be closed, and which can be recovered and repositioned if necessary or if the occlusion device has been positioned incorrectly.

Occlusion devices for undesired openings with means made of metal in the human body are known from U.S. Pat. No. 5,846,261, DE 695 34 505 or U.S. Pat. No. 5,725,552. In these previously known occlusion devices, either one or two clips for the wires and strands of the metal braid are present, which protrude into the blood stream, and, for fixing to a catheter, a thread is necessary, which makes it difficult especially to recover the device if the latter has been positioned incorrectly.

This latter disadvantage also applies to the occlusion element according to DE 2008 022 673 A1. Although the clips or holders for the filaments or wires of the braid face one another, they have to be screwed together, which entails additional effort.

### SUMMARY

It is therefore the object of the invention to make available an occlusion device for undesired openings in the heart, with two substantially parallel occlusion disks or umbrellas, whereby a reliable closure of an ASD or PFO or VSB of the heart is permitted without a metal clip, holder or threaded sleeve protruding into the blood stream.

This object is achieved by the means and features of the invention.

To achieve the object, provision is made that the spaced-apart umbrellas or occlusion disks are welded together at the mutually facing sides, i.e. no clips or holders or staples are needed to secure the wires of the braids. Accordingly, this also results in an occlusion device with two occlusion disks which are connected flexibly to each other, since no clips or staples or holders that have to be screwed together are provided between them, and this also leads to a reduced weight.

By virtue of its high degree of flexibility and low weight, an occlusion device of this kind can also be pulled back from a possibly incorrect position and thus recovered, and it can then be repositioned.

This results, however, in a reliable occlusion device for an undesired opening in the heart, which device can, if appropriate, also be used in other openings in the human body, for example without metal staples, holders or threaded sleeves protruding into the blood stream of the atria of such a heart with such an occlusion device, and possible repositioning can still be easily carried out.

In an expedient embodiment of the invention, provision can be made that the metal wires of the braids forming the umbrellas or occlusion disks are welded together at the mutually facing sides of the umbrellas or occlusion disks. The weld point is thus located in the area with the reduced radial dimension between the two occlusion disks, which area is arranged, in the position of use, in the opening that is to be closed, as is also known from DE 10 2008 022 673 A1, where however the two screwable holders of the two occlusion disks are arranged in this area of reduced radial dimension.

The material of the welded umbrellas or occlusion disks

and the braid forming them can consist of Nitinol wire, which has proven useful in medical technology.

Generally, as is known from DE 10 2008 022 673 A1, the two umbrellas or occlusion disks arranged at a distance from each other are approximately of the same size and of corresponding shape, for example discus-shaped. However, a modified arrangement can be provided in which the two umbrellas or occlusion disks arranged at a distance from each other have different sizes and/or shapes. In this way, they can be adapted to corresponding anatomical circumstances, particularly during their production.

In an expedient embodiment of the invention, provision can be made that, on each umbrella or each occlusion disk, several wires of the braids are intertwined, and intertwined wires of one occlusion disk are welded to those of the other occlusion disk. Since the wires of such occlusion devices and occlusion disks are chosen to be thin, welding of these wires is easily possible, particularly if the weld point formed by intertwined wires has a slightly greater cross section compared to that of a single wire.

As has already been mentioned, the connection between the umbrellas or occlusion disks is expediently and advantageously free of staples, holders or threaded sleeves and is formed directly by the weld. This connection of the two disks is very strong, but it is also flexible and can thus be easily adapted to the anatomy in the region of the opening that is to be closed.

In a further embodiment of the invention, provision can be made that the welded connection or the site of the welding of the intertwined wires is encapsulated by curable adhesive and/or flexible plastic. The simplest scenario is one in which the whole connection between the two occlusion disks has such an encapsulation which, by virtue of its connection to the wires and weld points enclosed by it, increases their tensile strength, fatigue strength and tear strength.

In a further embodiment of the invention, particularly for improving the seal, provision can be made that a plastic disk is integrated in the umbrella or in the occlusion disk for at least one of the atria. For example, a plastic disk of this kind providing additional sealing can be integrated in the occlusion disk for the right atrium of a heart, where the blood pressure is lower than in the left atrium. However, cases can also arise where a plastic disk of this kind providing additional sealing is integrated in the occlusion disk for the left atrium, and, finally, plastic disks could also be provided in both occlusion disks.

The occlusion disk or the umbrella that contains the plastic disk in the position of use can be open in some areas for the insertion thereof. Thus, in the braid, a specific insertion opening can be kept free from the wires, so as to be able to insert the plastic disk through such an opening.

In an embodiment of the invention that is of very considerable importance and that is also particularly advantageous for solving the partial problem concerning repositioning, provision can be made that a filament or double filament leading to the outside is provided on the right-hand umbrella in the position of use, or on the right-hand occlusion disk of the occlusion device according to the invention. In order to recover the occlusion device when it has been incorrectly positioned, it therefore suffices to exert a tensile force on this filament or double filament so as to bring the occlusion device back to the outside. Thereafter, it can be repositioned in a known and customary manner, with the user taking care to ensure improved positioning.

It is useful here if the filament or double filament comprises or engages around several wires of the braid. The tensile force of the filament or double filament can accordingly be easily exerted on the braid and therefore on the occlusion device, without the structure of the occlusion device or of its braid being adversely changed.

### BRIEF DESCRIPTION OF THE DRAWINGS

Illustrative embodiments of the invention are described in more detail below with reference to the schematic views in the drawing, in which:

FIG. 1 shows two examples of known occlusion devices for undesired openings in the heart, each of them with two approximately parallel umbrellas or occlusion disks which are arranged at a distance from each other and are made from a metal braid, wherein, on at least one face or on both faces, a clip or staple is provided on the outside for holding the wires of the braid,

FIG. 2 shows an embodiment of an occlusion device according to the invention for undesired openings in the heart, with two occlusion disks which are arranged at a distance from each other and are each formed from a braid, wherein the mutually facing sides of the braids are welded together without staples, holders or threaded sleeves,

FIG. 3 shows a modified embodiment of the occlusion device according to the invention, with two occlusion disks which are arranged at a distance from each other and are each formed from a braid, wherein the occlusion disk for the right atrium is partially open and contains a plastic disk inserted through this opening,

FIG. 4 shows a modified embodiment of the occlusion device according to the invention, with two occlusion disks which are arranged at a distance from each other, are formed from a braid, and have different shapes and/or sizes, firstly in a side view directed toward the narrow sides of the two parallel occlusion disks, and secondly in a plan view of one occlusion disk and of the second occlusion disk located behind the first one at this viewing angle,

FIG. 5 shows an embodiment of the occlusion device according to the invention, with two occlusion disks which are arranged at a distance from each other, are formed from a braid and, in the area where they are connected to each other, have a plurality of welded connections, and

FIG. 6 shows an embodiment of the occlusion device according to the invention with two occlusion disks, wherein an altered or modified disk shape is provided for the left atrium.

### DETAILED DESCRIPTION OF THE PREFERRED EMBODIMENTS

In the following description of the various embodiments, parts that correspond in terms of their function are given corresponding reference numbers even if they have a modified design.

An occlusion device for undesired openings in a human heart (ASD, PFO, VSD) is designated in its entirety by 100 and has two flexible umbrellas which are arranged at a distance from each other, serve as occlusion disks 3 and are made from a braid of metal wires 2, whereby openings in the human heart can be closed for example in the manner known from DE 10 2008 022 673 A1 and also from the prior art cited therein.

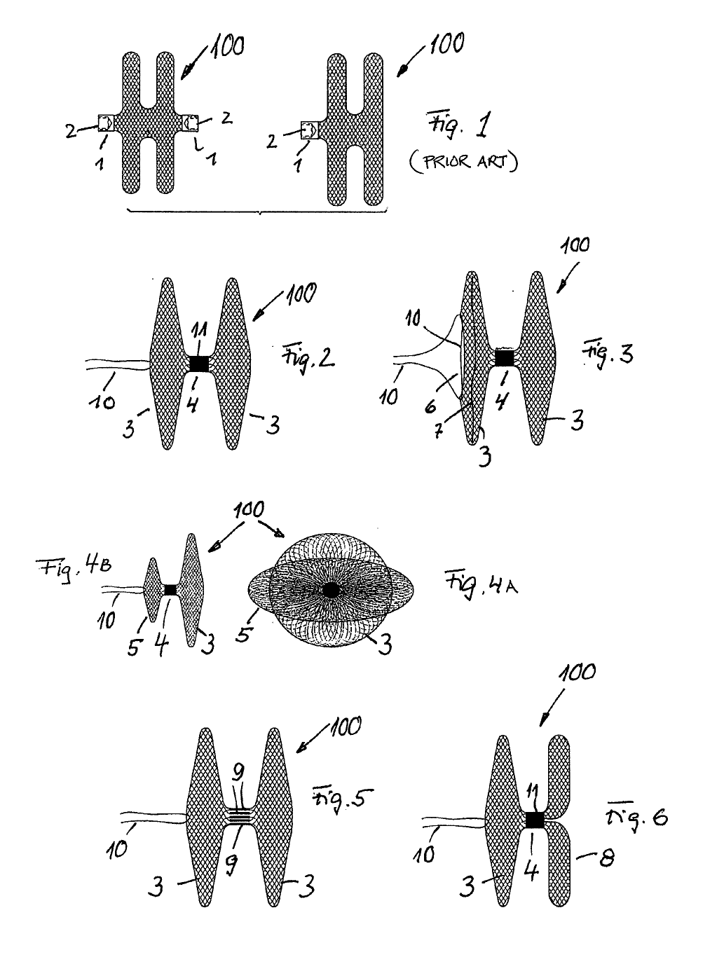

The occlusion devices 100 according to FIG. 1 have clips 1 and threads 2 which, in the implanted state, protrude into the atria of the heart.

By contrast, the occlusion devices 100 according to the invention as shown in FIGS. 2 to 6 are designed such that clips 1 and threads 2 of this kind are avoided.

FIG. 2 shows an embodiment of the occlusion device 100 with two approximately parallel occlusion disks 3 which are arranged at a distance from each other and are formed from a braid, wherein the braids and wires are welded to each other at the site of the smallest radial dimension between the two occlusion disks 3. This welding of the wires of the braids is identified by reference number 4.

FIG. 2 moreover shows, as also do FIGS. 3 to 6, a double filament 10 which engages on the right-hand occlusion disk 3 in the position of use and serves to recover or reposition the occlusion device 100. With this double filament 10, the user is able to apply a tensile force to the occlusion device 100 and, if appropriate, completely remove the latter and insert it anew in a known manner if it was initially positioned incorrectly.

It will be noted that this double filament 10 can comprise several wires of the braid, in order to ensure that the tensile force to be applied can be introduced easily and without structural changes of the occlusion device 10.

FIG. 3 shows an occlusion device 100 in which the occlusion disk 3 for the right atrium has an opening 6 in its braid. Since the blood pressure in the right atrium is always lower than in the left atrium, the umbrella structure there, i.e. the structure of the umbrella or of the occlusion disk 3, can be designed such that a thin plastic disk 7 can be integrated for sealing purposes, as is indicated in FIG. 3. This plastic disk 7 can be inserted through the opening 6. By virtue of the opening 6, the double filament 10 has a wider engagement on this occlusion disk 3 in the edge area of the opening 6.

FIG. 4 shows a modified embodiment of the occlusion device 100 in two different views. The view on the left corresponds to the views according to the other figures, it being clear that an occlusion disk 5 can be provided that is different in terms of its shape from the shape of the occlusion disk 3. If one considers only the left-hand part of FIG. 4, the occlusion disk 5 as a whole can be smaller, for example once again with an approximately circular shape as in the case of the other occlusion disks 3. However, the right-hand side of FIG. 4 shows that, in this case, the occlusion disk 5 has an approximately elliptic shape and in this way differs from the circular shape of the occlusion disk 3. It is thus possible for different anatomical circumstances to be taken into account.

FIG. 5 shows an expedient embodiment of the occlusion device 100, in which the wires of the braid of the two occlusion disks 3 are connected by several welds 9 in the area between these two occlusion disks 3. These several welds 9 are indicated by parallel lines in this connection area between the two occlusion disks 3.

FIG. 6 shows an embodiment of the occlusion device 100 in which one of the two spaced-apart occlusion disks 3 has a modified disk shape, and this modified occlusion disk 8 is intended for the left atrium. The weld 4 according to FIGS. 2 to 4 is once again provided.

The welded connection 4 or the several welds 9 can be encapsulated by curable adhesive and/or flexible plastic. The weld point 4 shown in each of the drawings can be understood in the sense that its black area represents an encapsulation 11 of the weld point 4 or, if appropriate, also of individual welds 9.

The occlusion device 100 for undesired openings in the heart (ASD, PFO, VSD) has two flexible occlusion disks 3 which are arranged at a distance from each other and are formed from a braid of metal wires. It is thus possible, especially in the human heart for example, to occlude openings that are present between two atria, and the occlusion device 100 can also be recovered and repositioned if necessary or if the occlusion device has been positioned incorrectly. In the region of the smallest radial extent of the occlusion device 100, the mutually facing parts of the occlusion disks are connected to each other by welding, such that clips, staples and/or threads are avoided.

1. An occlusion device (100) for undesired openings in a heart (ASD, PFO, VSD), comprising two flexible and welded umbrellas, which are arranged at a distance from each other, that serve as occlusion disks (3) and are made from a braid of metal wires, and are adapted to close openings in a human heart, and which are recoverable and repositionable if necessary or if the occlusion device has been positioned incorrectly.

2. The occlusion device as claimed in claim 1, wherein the metal wires of the braids forming the umbrellas or occlusion disks (3) are welded together (4) at mutually facing sides of the umbrellas or occlusion disks (3).

3. The occlusion device as claimed in claim 1, wherein the material of the welded umbrellas or occlusion disks (3) and the braid forming them consists of Nitinol wire.

4. The occlusion device as claimed in claim 1, wherein the two umbrellas or occlusion disks (3; 5) arranged at a distance from each other have at least one of different sizes or shapes.

5. The occlusion device as claimed in claim 1, wherein on each of the umbrellas or each of the occlusion disks (3; 5), several of the wires of the braids are intertwined, and the intertwined wires of one occlusion disk are welded to those of the other occlusion disk.

6. The occlusion device as claimed in claim 5, wherein a connection between the umbrellas or occlusion disks (3) is free of staples, holders or threaded sleeves and is formed directly by a welded connection (4).

7. The occlusion device as claimed in claim 6, wherein the welded connection (4) or a site of the welded connection of the intertwined wires is encapsulated by at least one of curable adhesive or flexible plastic (11).

8. The occlusion device as claimed in claim 1, wherein a plastic disk (7) is integrated in the umbrella or the occlusion disk (3) for at least one atria.

9. The occlusion device as claimed in claim 8, wherein the occlusion disk (3) or the umbrella that contains the plastic disk (7) is open in some areas for insertion thereof.

10. The occlusion device as claimed in claim 1, wherein a filament or double filament (10) leading to outside is provided on a right-hand one of the umbrellas in a position of use or on the occlusion disk (3) arranged on the right.

11. The occlusion device as claimed in claim 10, wherein the filament or double filament (10) comprises or engages around several of the wires of the braid.
